(19) United States
(12) Patent Application Publication
(10) Pub. No.: US 2008/0216844 A1
(43) Pub. Date: Sep. 11, 2008

(54) STERILE DRAPING FOR THE BORE OF A MEDICAL IMAGING SYSTEM

(76) Inventors: Cheryl Olfert, Winnipeg (CA); Lindley Ward, Winnipeg (CA); Victoria D.M. Hornblower, Winnipeg (CA); Stephen G. Hushek, Wauwatosa, WI (US); Michael E. Pilon, Winnipeg (CA); Edward Stewart Richmond, Winnipeg (CA); Mauricio C. Ede, Winnipeg (CA); Garry Bistyak, Lorette (CA); Gregory P. Mallett, Winnipeg (CA); C. Hermil A. Moreno, Winnipeg (CA)

Correspondence Address: ADE & COMPANY INC., 2157 Henderson Highway, WINNIPEG, MB, R2G1P9, CA

(21) Appl. No.: 11/944,514

(22) Filed: Nov. 23, 2007

Related U.S. Application Data
(60) Provisional application No. 60/867,141, filed on Nov. 24, 2006.
(60) Provisional application No. 60/979,424, filed on Oct. 12, 2007.

(51) Int. Cl. A61B 19/12

(52) U.S. Cl. 128/856

(57) ABSTRACT

A sterile drape for use with a magnet of a magnetic resonance imaging is formed from a generally cylindrical sheet of a fabric. This is collapsible into an annulus and can be expanded axially to line the interior of the bore of the magnet. End flaps can close the ends of the bore and folded outwardly to cover the end faces of the magnet. The sheet can be supported by a wire or other support and can be stored on the magnet and end covers or in a ceiling suspended container.

This application claims the benefit under 35 U.S.C. 119 of the filing date of the Provisional Application Ser. No. 60/887,141 filed Nov. 24, 2006 and of the filing date of the Provisional Application Ser. No. 60/979,424 filed Oct. 12, 2007.

This invention relates to a drape for a medical device having a bore for patient entry, which is particularly but not exclusively related to imaging systems and MRI systems in particular, but can also be applied to any medical system that has a bore for patient entry.

### BACKGROUND OF THE INVENTION

Imaging systems, such as magnetic resonance imaging (MRI) and computed tomography (CT), which were originally developed for diagnostic purposes, are now increasingly used for surgical, intra-operative, and interventional applications. Thus, there is often a need to provide a sterile environment for the patient. Typically, the medical imaging device cannot be sterilized; to maintain a sterile environment for the patient, the interface between the sterile and non-sterile areas is obtained by a series of drapes.

A current arrangement for attaining a sterile surgical and patient environment during imaging (e.g. MRI) is by placing two drapes over the head of the patient and pulling a sterile draping bag down over the patient's body. For example, if neurosurgery is being performed, the patient is entering headfirst into the imaging system, and therefore the sterile drape is being pulled from the head towards the feet, covering the table and patient. Currently, the present assignee IMRIS incorporated provides a moving MRI system as shown in U.S. Pat. No. 5,735,278 (Hoult) filed Mar. 15, 1996. The MRI device enters the room or table area under operator guidance and images the body part of interest and after which, the MRI device exits the room or table area. Finally the sterile drape is removed from the patient and the surgery resumes. In this manner, the surgical field is kept sterile even though the MRI is not sterile.

Draping of parts of a magnet for MRI usage is shown by Kimberley-Clark, U.S. Pat. No. 6,105,578 (Sommers) issued Aug. 22, 2000 entitled “Equipment drape for use with an interventional magnetic resonance imaging device”. In this patent, proposed for use in the GE double-donut MRI design, they discuss the usage of one or more U-shaped drapes that hang between the two MRI magnets, with the patient being in the middle.

Draping of a C-arm X-ray device is shown by U.S. Pat. No. 7,044,132 (Masini) issued May 16, 2006 entitled “Surgical drape adapted for use with radiological equipment”. This patent outlines the usage of a surgical drape having pleats or a gathered section, or with a tunnel or tent formed in the material, which will allow for movement of the C-arm X-ray device, without displacing the surgical drape and disturbing the sterile field.

U.S. Pat. No. 7,096,871 (Lee) issued Aug. 29, 2007 shows a drape which has a pouch opened by an inflated tube around the rim.

Canadian Application 2,265,805 (Asmus) assigned to 3M and published as a PCT publication on Apr. 23, 1997 discloses a method for dispensing discrete lengths of surgical drape material from a supply of the same.

U.S. Pat. No. 5,617,027 (Decke) issued Apr. 1, 1997 shows a vacuum pillow in conjunction with a local antenna for use with nuclear magnetic resonance diagnostics. The vacuum pillow is made from a flexible material and contains small polystyrene balls and a vacuum line. The vacuum pillow is flexible to allow the local antenna to be positioned around the patient; upon application of the vacuum, the pillow becomes rigid and provides stability and support to the local antenna.

### SUMMARY OF THE INVENTION

It is one object of the invention to provide an improved drape and an improved method of providing a sterile environment for a medical system. Although the invention will be described with reference to an MRI scanner, the invention is applicable to any medical system having a bore or tube for patient entry and subsequent imaging or patient treatment.

According to the invention there is provided a sterile drape assembly for use with a medical device having a cylindrical bore, the drape comprising:

a sheet of a surgical drape material;

a mounting arrangement for supporting the sheet in a generally cylindrical shape or part cylindrical shape as a sleeve within the cylindrical bore of the magnet;

the surgical drape material having the characteristics of being sterile and compatible with the medical device.

In one arrangement, the sleeve is shaped and arranged such that it is formed into an annular structure which can be deployed by expanding the structure axially along the bore. The annular structure can be supplied in an annular container and can include pull strings such that the sleeve is deployed by the pull strings extending along the bore. The sleeve can also be deployed by providing a first end for engaging an end of the bore of the magnet and a second end for engaging a diagnostic table such that relative movement between the magnet and the diagnostic table acts to deploy the sleeve.

In one embodiment the annular structure may include a wire support system which can be deployed axially and which provides a support for the sleeve.

In one embodiment the annular structure may include an axially extending arm such that the sleeve is deployed by axial extension of the arm.

In one embodiment the annular structure may include adhesive strips along an outside surface for bonding to the bore which are covered by cover strips and the cover strips are arranged such that longitudinal pulling of the cover strips acts to deploy the sleeve.

Where the annular structure is supplied in an annular container, the container may form part of an end collision detection system on an end of the magnet.

Where the annular structure is supplied in an annular container, the container may be carried on a support not attached to the magnet but is available to be moved into place at the end of the magnet.

In another embodiment, the sleeve is shaped and arranged such that it is rolled lengthwise into a cylindrical structure with an axis which is arranged to extend along the bore and which can be deployed by expanding the structure outwardly into a cylinder shape. In this embodiment, the sleeve may be formed from a resilient sheet material which has sufficient resilience to expand from the initial rolled position to an expanded cylindrical condition.

In another embodiment, the sleeve may be rolled about a non-magnetic rod. The drape is then deployed by rotating the non-magnetic rod along the circumference of the bore.

While in some cases the sleeve may be self supporting or attached by adhesive or other connections, in another arrangement there is provided a cylindrical support member arranged to be mounted within the bore for supporting the sleeve. In one construction, the cylindrical support member is hollow and includes a vacuum line which is connected to a hollow interior of the support member for drawing the sleeve against a perforated inner surface of the support member. In another construction the support member is arranged to engage the sleeve by an elastic nature of the sheet which is wrapped around the support member. Additionally, the elastic sheet may be attached directly to the front and back faces of the magnet, by adhesives or clips. The cylindrical support member may be defined by a series of spaced members such as a wire mesh.

The cylindrical support member may be a single elongated piece or may include a plurality of sections at axially spaced locations which can expand axially by relative movement of the sections.

The cylindrical support member may include two support components each associated with a respective end of the bore for insertion into the respective end.

In yet a further alternative, the sleeve is formed from a stiff material which is shaped and arranged such that it is supplied in an originally folded and collapsed condition from which it can be expanded to form the cylindrical shape by unfolding.

In another embodiment the sleeve, is made from a soft, pliable material that contains a single chamber, or a series of semi-partitioned chambers, and may be inflated into a semi-rigid cylinder through the use of pressurized air or gas.

In another embodiment the sleeve, is made from soft, pliable material that contains either a single chamber or a series of semi-partitioned chambers may be filled with small solid objects (example, polystyrene balls); and a vacuum may then be applied to the chambers, removing all air between the small solid objects and causing the sleeve to become semi-rigid.

For further advantage, each of the above arrangements may include a construction where the sleeve includes a plurality of overlying layers that may be peeled away one after the other to reveal the next underlying sterile layer after a first is used.

For further advantage, each of the above arrangements may include a construction where the sleeve is formed from a first material which is relatively stiff to conform to cylindrical portions of the bore and includes portions of a more flexible material which accommodate projections and/or recesses in the bore.

For further advantage, each of the above arrangements may include a construction where the sleeve has circular portions defining sealed ends of the sleeve. The sealed ends may be arranged to be removable prior to introduction of the patient into the bore or the sealed ends may be formed in separate pieces which can be folded back around the sleeve to engage and cover an end face (or portion thereof) of the magnet. After the patient has been removed, additional end covers may also be attached, through adhesives, in order to maintain sterility of the bore for additional imaging sessions, for the same patient.

Thus sterility within the magnet of the MRI system, as proposed herein, is maintained by forming a drape within the magnet instead of draping the patient. This method achieves improved patient access, reduces need to move surgical equipment that is used for the surgery, and potentially allows a faster time between surgery and imaging steps. Patient safety is also increased. In addition, because the magnet is of constant size whereas patients are of various sizes, there is an increased ability to fit the drape to the MRI, ensuring the optimum in sterility for the patient. As well, through the method of draping the MRI, the sterile space is increased in comparison to patient draping techniques. This increase in sterile space can be important as surgical robots may be used in the bore of the MRI to perform surgery, in cases where a portion or the entire robot is also part of the sterile field.

For an MRI system the patient must be imaged inside the bore of the magnet. Thus the drape is the size of the diameter of the bore of the magnet.

In one arrangement it can be placed at the end of the bore where the patient table enters with double sided adhesive or some other suitable connection method. A string or cord is on the far end of the drape to allow the nurse or technician to grab the cord and pull it through the bore as the drape expands (accordion or slinky style) through the bore. Either end or both ends of the drape can have flaps that fold into the center of the drape maintaining a sterile environment through the center of the drape. These flaps can then fold out and attach with adhesive to the face of the machine (covering a portion of the face of the machine and making this portion a sterile surface) just prior to the patient table entering.

The support for the sleeve can be provided by a helical wire that can be pulled from a collapsed annulus to a helical shape to cover the bore of the magnet. The helical wire design approach is only one method of achieving structural rigidity so that the drape stays off of the patient. Alternative approaches to move or roll the drape can include air pressure methods within the bore of the MRI, integrated cabling within the bore of the MRI, or various types of mesh designs. In some cases having adhesive attachment at both ends of the bore, and pulling the drape tightly against the bore successfully maintains the drape away from the patient.

This approach to sterility can work with any sized MRI bore, whether it is the 70 cm. bore associated with the Siemens Espree MRI or the 60 cm. bore of other magnets, Clearly, there are various specific drape designs that can achieve the same basic purpose. This approach works with cylindrical bore magnet designs that are open on both ends or capped at one end. Whereas with uncapped designs the nurse can pull the cord from the far end of the MRI, with capped designs an articulated arm can be used to pull or push the cord to the end of the closed bore. This design will also work with various styles of uncapped magnet designs, by applying the drapes inside the magnet bore instead of draping the patient.

The presence of the draping does not affect the patient services that are available in the bore, because the surgical patients are typically not conscious during the operation. However, for cases where the patient is conscious (e.g. interventional), the drape may have various designs printed on its surface that will make the bore more aesthetically pleasing and have a calming affect on the patient. This is especially important for pediatric cases. Various mechanisms can be used to roll out the drape which may be more or less complex, depending on the customer requirement and the type of design that is delivered.

For example, the drape may be manufactured integral to a magnet collision or anti-collision system that is mounted on one end face of the magnet bore. Such an anti-collision device may be of the type disclosed in co-pending U.S. application Ser. No. 11/397,910, filed Apr. 5, 2006 which corresponds to Canadian Application Serial No: 2,542,932, filed Apr. 41, 2006, the disclosure of which is incorporated herein by reference.

Alternatively, the drape may be manufactured and designed so that it fits within the end covers of the magnet. A third alternative is that the drape is attached by the surgical staff soon after the surgery begins. A further alternative is that the drape mechanism is suspended from the ceiling in a storage compartment and when not in use is retracted toward the ceiling.

### BRIEF DESCRIPTION OF THE DRAWINGS

One embodiment of the invention will now be described in conjunction with the accompanying drawings in which:

FIG. 1 is a side view of a magnet including a sterile draping system according to the present invention.

FIG. 2 is a side view of embodiment of the drape in FIG. 1 in the expanded condition.

FIG. 2A is an isometric view of embodiment of the drape of FIG. 2 in the expanded condition.

FIG. 3 is a side view of an alternative form of the drape of FIG. 1.

FIG. 3A is an isometric view of the drape of FIG. 3.

FIG. 4 is a first isometric view of a further embodiment of the drape in FIG. 1 FIG. 4A a second isometric view of the further embodiment of the drape in FIG. 4.

FIG. 5 is an isometric view of a further embodiment of drape according to the present invention.

FIG. 6 is a side view of an alternative embodiment of the drape in FIG. 1.

FIG. 6A is an isometric of the alternative embodiment of the drape in FIG. 6.

FIG. 7 is a side view of a further embodiment of the drape according to the present invention.

FIG. 7A is an isometric view of the further embodiment of FIG. 7.

FIG. 8 is a side view of a further embodiment of the drape according to the present invention.

FIG. 8A is an isometric view of the embodiment of FIG. 8.

In the drawings like characters of reference indicate corresponding parts in the different figures.

### DETAILED DESCRIPTION

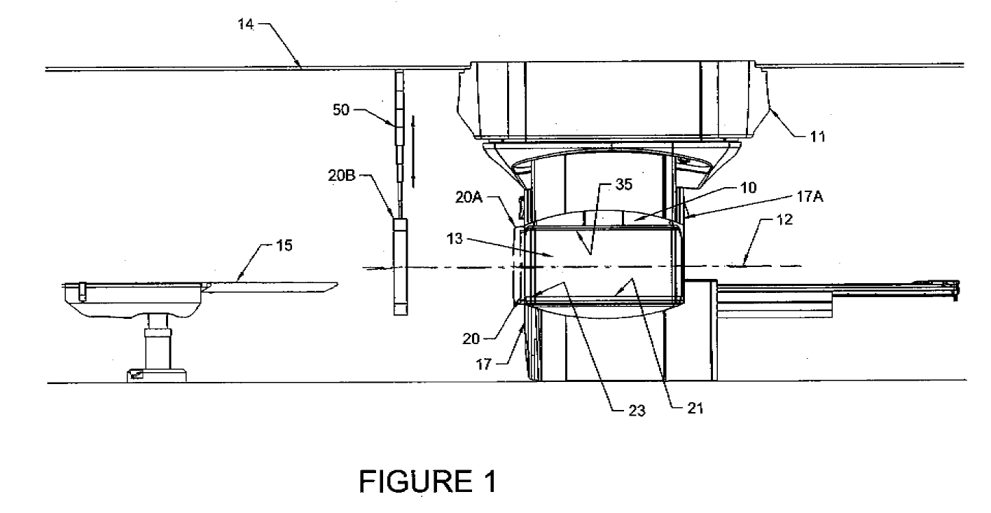

In FIG. 1 is shown a magnet of a magnetic resonance imaging system where the magnet is indicated at 10 and is of the cylindrical type defining a bore 13 surrounding an axis 12. The magnet is mounted on a support frame 11 carried on a track 14 for the movement of the magnet along its axis 12. The magnet is associated with an operating table 15 so that the magnet can be moved to a position surrounding the operating table at stages during an operating process to obtain images on an intra-operative basis.

Further details of this arrangement are shown and described in the above mentioned United States patent assigned to IMRIS, the disclosure which incorporated herein by reference.

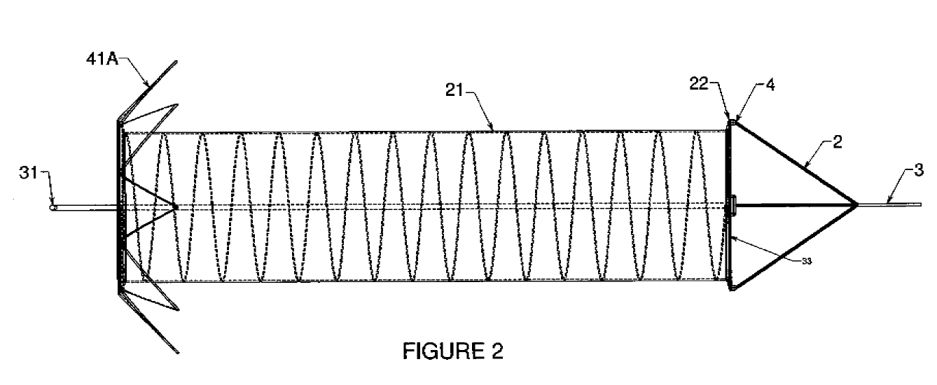

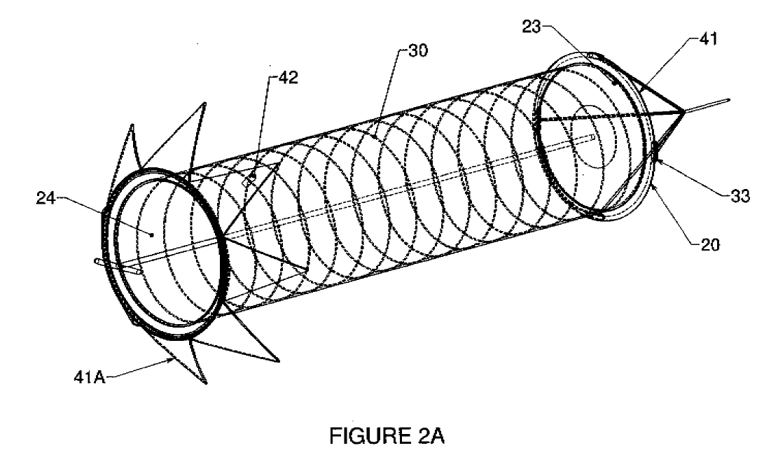

In FIG. 2 is shown the drape which is used in conjunction with the magnet 10. The drape forms when expanded a cylindrical sleeve-shaped structure 21. This has two open ends 23 and 24. The sleeve is collapsed axially (not shown) to form an annular structure which is housed within an annular container 20. The annular structure 22 is thus protected and contained within the annular container when not in use. The annular container can be opened and the drape therein exposed so as to be expanded into the cylindrical condition. The housing or container 20 as shown in FIG. 1 is mounted on the end face 17 of the magnet.

In FIG. 2, pull strings 2 are shown which include a longitudinal string 3 which extends along the axis together with diverging pull strings 2 which extends along the axis to the end 24 of the cylindrical sleeve. In order therefore that the sleeve is pulled through the bore of the magnet, the string or cord 3 can be pulled through the magnet allowing the user to pull on the ends of the sleeve drawing it through the bore to the far end 17A of the bore opposite the end face 17.

In an alternative technique for deploying the annular drape, for use with a magnet having a diagnostic table 18 mounted thereon, one end of the drape is attached to the bore-entry end of the diagnostic table and the other end of the drape is secured to the opening of the bore that is furthest from the diagnostic table, 17. The drape deployment process begins with positioning the diagnostic table 18 so that it is within the bore of the magnet. Once the ends of the surgical drape have been secured, the diagnostic table is then moved so that it is completely outside of the bore. This acts to deploy the drape along the length of the bore.

The housing 22 is mounted on the end wall 17 of the magnet by attachments 4 which can be adhesive or of the hook and loop-type.

The sleeve as shown in FIG. 2 is supported closely adjacent the internal surface of the bore by a helical wire 30. The helical wire thus can be collapsed into the annular space of the housing 20. However when pulled it expands axially by the turns of the helix increasing in spacing while providing a support for fabric holding it away from the axis 12 to the wall of the bore.

In FIG. 2 the pulling system is used with a bore of a magnet which is open at each end allowing the user to reach into the cord 3. If one of the ends of the magnet is closed, an arm 31 is provided which grasps the end 23 of the sleeve and pushes it along the bore from the open end at the end wall 17 up to the closed end. In this embodiment the end of the sleeve can be closed by an end sheet 33 which is circular and closes the end 23.

In FIG. 1 there is shown an arrangement in which the sleeve extends to the far end of the bore and is supported on the interior of the bore by a suitable generally cylindrical support member indicated at 35. This can be a mesh, or can be guide wires which are mounted on the inside surface of the bore and which hold the sleeve as it is expanded along the bore.

Alternative arrangements can be provided for mounting the sleeve on the interior of the bore. This can simply be done by attaching elements at one end 17, by pulling the sleeve along the bore and by attaching similar elements at the opposed end 17A of the bore thus pulling the sleeve tight within the bore with the elements holding the cylindrical portion of the sleeve in contact with the inside surface of the bore.

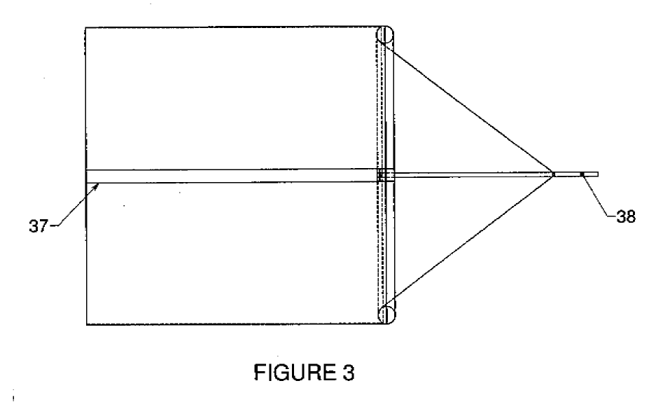

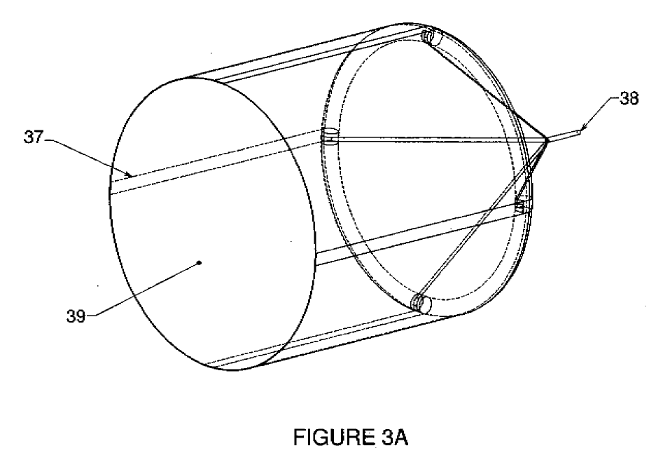

As a further alternative arrangement to secure the drape shown in FIG. 3, the outer surface of the drape has strips 37 of adhesive that secure the drape to the inner surface of the bore. The inner surface of the drape remains sterile as the drape is unrolled. The adhesive may be coupled with cover strips 38 of a non-adhering material along the length of the drape to prevent the adhesive from sticking to itself. The cover strips may also serve as a means of unraveling the drape and as the cover strips are pulled, the drape unravels and may be pressed against the side of the bore.

To maintain sterility, a top layer or cover sheet of material 39 for example of a plastic film may be present during drape installation, but removed immediately prior to patient entry into the bore.

The container 20 in FIG. 1 which contains the drape can comprise a separate container merely mounted on the end face 17. However in an alternative arrangement the container may be located within end covers on the magnet so that it is held in place and kept available for use when the surgical drape is required. Yet further the container 20 may be contained within or formed part of an end collision detection system 20A which forms an annular member on the end face of the magnet and includes sensor elements which are deformed on impact of the magnet end wall with any object such as the operating table or component thereof.

In FIG. 2 is shown a further option which includes with end flaps 41A each of which is shaped to form a segment of the end circle of the opening at the end of the bore. These flaps can be folded inwardly as shown at 41 to close the end of the sleeve while the magnet is moved into the theatre, to maintain a sterile environment with the bore. However when the magnet is in place, the same flaps 41 can be moved to the position as indicated at 41A where those flaps extend outwardly in the radial plane at the end of the sleeve 40 where they can be attached by fastening portions 42 to the end face of the magnet thus providing a coverage of the whole or part of the end face of the magnet. While the flaps are shown as only partial flap portions partially covering the end of the magnet, these can be shaped so that they can provide full coverage in an annulus surrounding the bore.

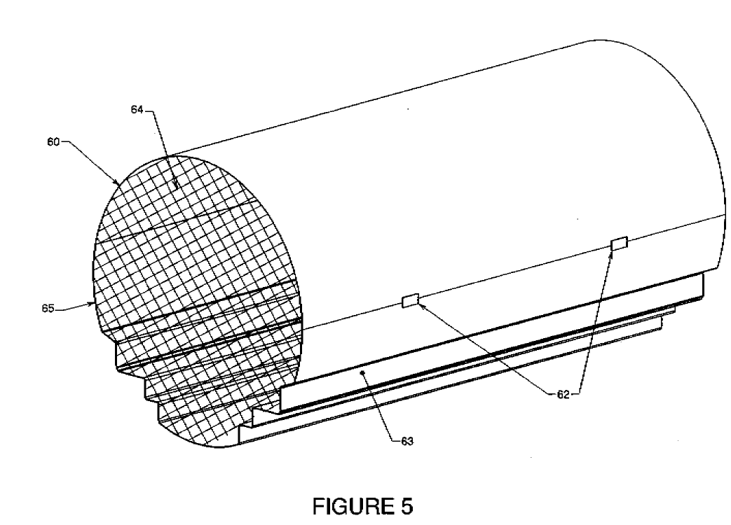

The drape may also have circular end covers 64 (FIG. 5) that close the end of the cylindrical drape. These can be attached to the drape using an adhesives or may be formed integrally with the drape but with a tear-away edge 65. The end covers are removed immediately prior to patient entry.

In FIG. 1 is shown a further alternative for mounting the container 20B in which the container of the drape is carried on a suspension system 50 carried from the ceiling or the track 14 on which the magnet runs. Thus the container is not attached to the magnet itself but is readily available to be moved into place between the magnet and the operating table 15 when the drape is required. The container 20B thus holds the drape in its annular collapsed condition so that it can be brought up to the end face 17 of the magnet and the drape expanded into the cylindrical condition to be passed through the bore of the magnet.

In an alternative embodiment the expanding drape consists of a flexible sheet of material that is rolled up lengthwise into a tube shape.

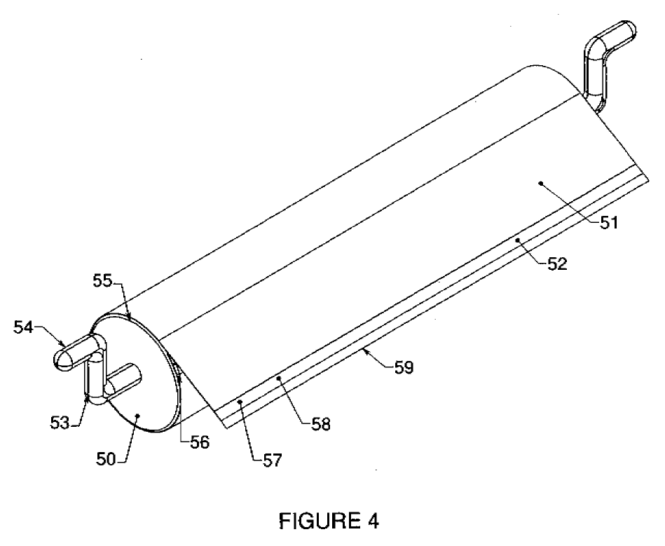

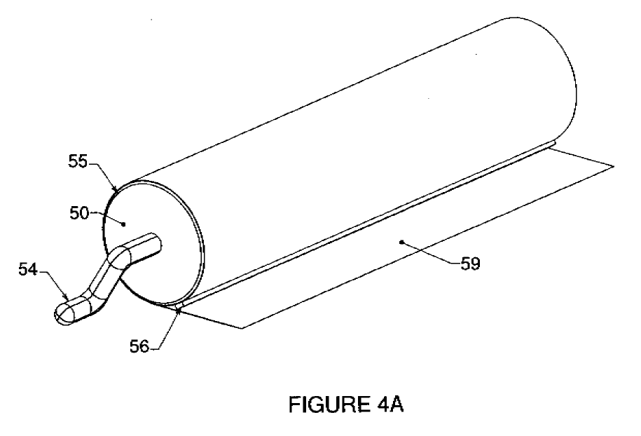

In one embodiment shown in FIG. 4, the sterile drape is formed of a flexible, flaccid sheet material with an adhesive coating 51 on the outer surface of the drape. The long edge of the drape 52 is applied to the inner surface of the bore, the drape is then unrolled and pressed against the inner surface of the bore. The drape can be coiled about a non-magnetic rod 53, having handles 54 on either end to facilitate unrolling the drape while maintaining sterility of the drape. The drape roll may also be encased in a canister 55 for ease of use. To further assist in the application of the drape, a track may be attached to the front and back faces of the magnet, which will guide the canister as it is being rolled along the inner surface of the bore. The canister may also have a pliable ridge or lip 56 along the edge of the exit of the drape, in order to press the drape into corners or ridges along the bore. The drape may be pre-cut to specific lengths required for draping of a particular bore diameter, or may be cut at a particular length after it has been applied to the bore by a cutting edge located on the canister.

In all embodiments described herein the drape may be formed as a multi-layer drape 57 that is installed in the bore with one application, but has layers 58 that may be peeled away after each clinical use, to reveal a sterile drape surface underneath.

In addition or as an alternative, the bore drape may also have a drape cover 59 on the inside surface, such as a plastic film, that may be easily removed immediately prior to imaging. This will ensure that the drape surface remains completely sterile for patient entry.

In another embodiment shown in FIG. 5, the drape is formed of a resilient sheet material 60 which has sufficient resilience to expand from the initial tightly coiled tube position to an expanded cylindrical condition matching the shape of the bore. The drape is placed within the bore, so that the axis of the drape is approximately parallel with the axis of the bore. The drape is then released and uncoils from the rolled condition to the cylindrical condition to fit the inner surface of the bore. The drape material must be flexible in order to coil up, but have the elasticity to spring back to an uncoiled shape, thus forming to the bore profile. The drape is held in the coiled condition by clips or by adhesive strips 62

For imaging systems with bores that do not have an inner surface which is entirely cylindrical, for example it has projections or cutouts in portions of the bore profile, such as ridges or rails within the bore the flexible sheet of material may be interspersed with portions 63 of a soft and pliable drape material at the required locations to cover the ridges or rails, so that the bore drape closely fits the contour of the bore profile. Thus the flexible sheet covers those portions of the bore having a smooth, arc contour, with the soft drape material portions 63 covering the portions of the bore with more detailed contours. The rolled drape may be deployed automatically, that is the drape remains tightly rolled by packaging so that once removed from the package, the drape expands by its own resilience. Alternatively the drape may be deployed by a manual action. Examples of manual deployment include an adhesive strip securing the outer, long edge of the drape to the outer surface of the drape, when the drape must be deployed, the adhesive strip may be manually removed. The drape may also be secured in a rolled position prior to deployment through the use of devices such as clips, which secure the ends of the roll drape. When the drape is deployed, the clips may be removed, either directly, or by pulling on cords attached to the clips.

The expandable roll drape may also incorporate covers or flaps 64 which seal both ends of the drape until patient entry. These covers or flaps may be manufactured from a soft, pliable material, which will expand with the drape as it is being deployed. Thus removable covers may be used which will completely seal the drape during deployment, and be removed immediately prior to patient entry. The covers or flaps as shown are tucked inside the drape when rolled up or may lie outside the drape prior to deployment.

The covers 64 may be completely removed immediately prior to patient entry into the bore. Thus, the drape may be completely installed in the imaging system, with only the end covers to be removed for patient imaging. These ends may be completely sealed to the drape by adhesive and easily peeled off when needed or the ends may also be attached to the drape though a “tear-away” edge 65 that allow them to easily be torn from the drape. Adhesive end covers may also be applied after imaging (i.e. patient has been removed from the bore but will be scanned again during the same procedure) to maintain sterility of the bore.

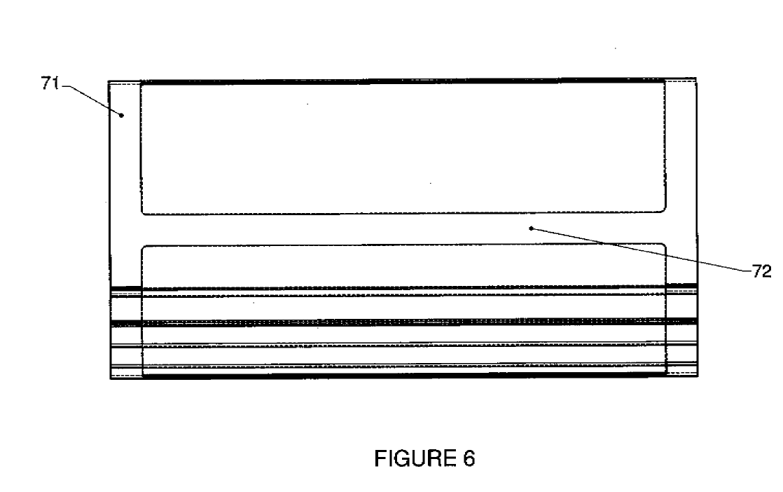

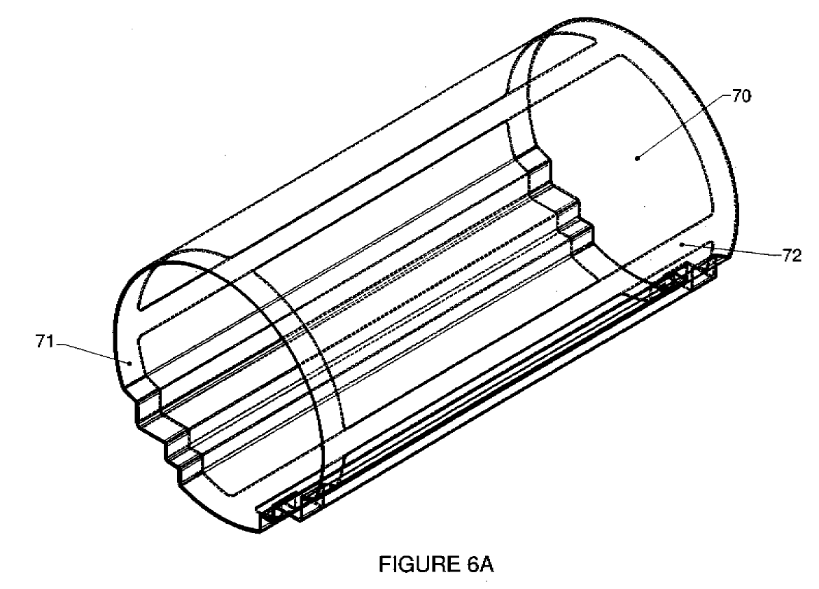

In FIG. 6 is shown a further embodiment where the drape is installed by an inflatable arrangement. The inflatable drape may consist of a soft, flexible material 70 that contains chamber(s), which may be filled with air or gas to inflate into a semi-rigid cylindrical structure. The drape may be comprised of a single chamber, continuous along the length of the bore, or it may have semi-partitioned chambers that form a cylindrical frame. In one embodiment support hoops 71 at the ends and ribs 72 extending along the length of the drape form the frame, but a spiral or other configurations could also be used.

The inflatable components of the drape may be inflated by a hand-operated air pump, by air lines within the operating room, or by a small carbon dioxide gas cylinder or cartridge (similar to those used in automatically inflating life vests). Prior to inflation, the drape may be extended along the length of the bore, whereby the drape expands radially during inflation, or the drape may be both extended axially and then expanded radially during the inflation process. A double-lumen, or two-way valve may be used for inflation. To minimize any risk of the drape deflating and collapsing on the patient, two independent inflation chambers within the drape may be used. Alternatively, the drape may be attached to the inner surface of the bore through means such as adhesives. The inflatable components of the drape may remain connected to the pressurized gas source for the duration of Or if the inflatable components can maintain pressure without a constant source the inflatable components may be sealed and isolated from the pressurized gas source once inflated.

In a similar embodiment to the inflatable drape, the sleeve may consist of a flexible material, having one continuous chamber or a series of semi-partitioned chambers, the chamber(s) being filled with small solid objects (for example polystyrene balls). The sleeve also contains a vacuum line and when a vacuum is applied, the sleeve transforms from a very flexible and flaccid object to one that is semi-rigid. In its flexible state, the sleeve or drape may be applied to the bore or surgical equipment, once positioned, the vacuum may be applied, giving the drape the stability and stiffness to maintain it's desired shape and position.

In a further embodiment an insert support sleeve is mounted in the bore and the drape attached to the support sleeve. The support sleeve is continuous and runs the entire length of the bore.

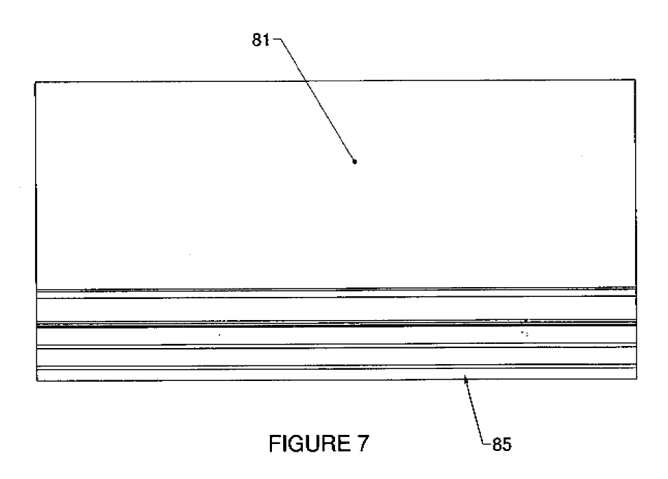

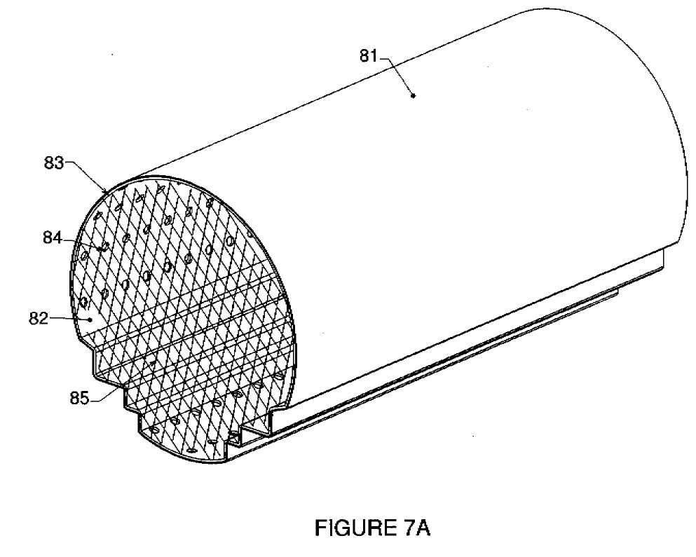

In a first option shown in FIG. 7, the support sleeve is hollow so that the sleeve has an outer wall 81 with the same profile as the bore, an inwardly facing wall 82 and having sealed ends 83. The inner wall of the sleeve has an array of small holes 84, and a vacuum line is connected to the hollow interior of the sleeve preferably at one end of the sleeve. A sterile lining 85 is then inserted into the bore and secured the lining to the inner surface of the sleeve by vacuum drawn through the holes into the sleeve.

In a second option (not shown), the sterile drape is attached to the support sleeve by an elastic nature of the drape. The support sleeve has an overhanging annular lip at one end that covers a portion of the front face of the imaging system. This lip provides a means of securing the drape to the support sleeve in that the drape is formed of an elastic material which is stretched over the lip of the sleeve. The support sleeve can be formed of two sleeve components, one inserted into each end of the bore.

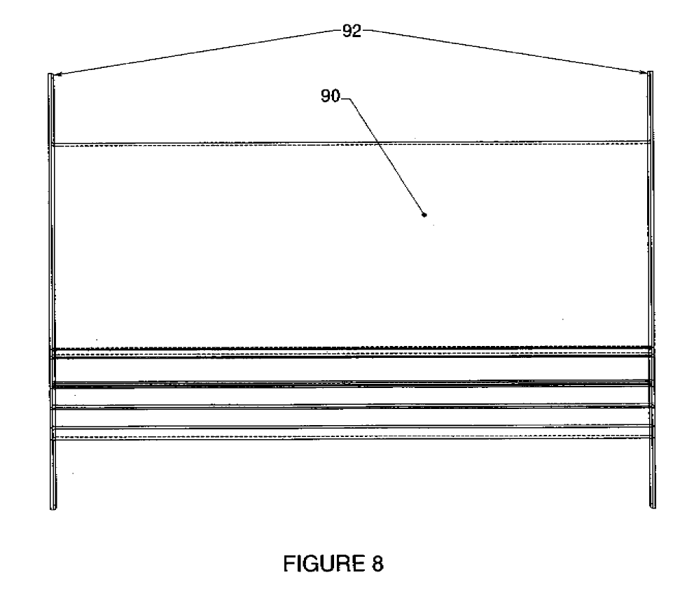

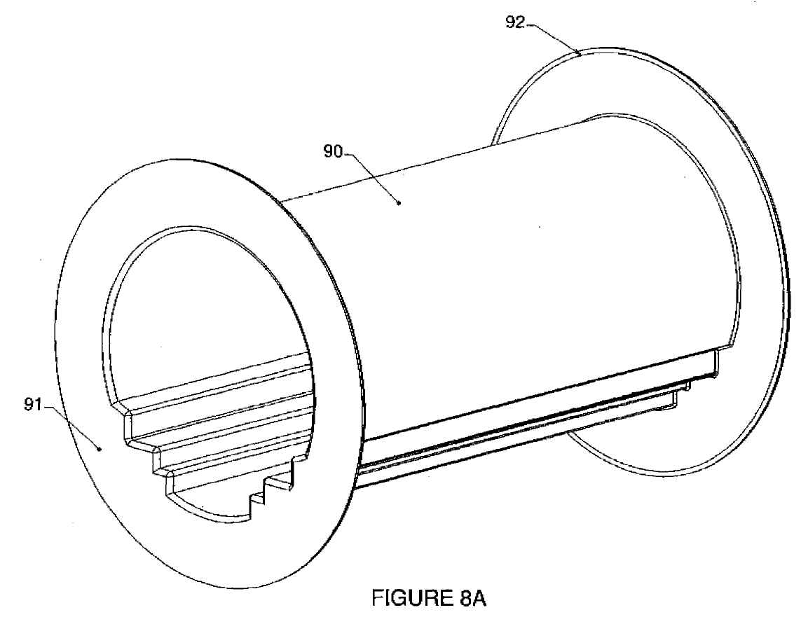

In FIG. 8, the elastic drape may also be attached directly to the magnet front and back faces by a series of clips or adhesive pieces. The bore surface is covered by sleeve 90, while the front and back faces of the magnet are covered by annular sections of drape 91. The annular sections have an elastic circumference 92 that is connected to the magnet covers through adhesives or clips.

In an alternative arrangement (not shown) the support sleeve is formed of a number of sections, which are arranged coaxially and arranged at axially spaced locations so that they can be expanded axially to the full length of the bore, and compressed to a relatively short length. The sections may be inserted one inside the next so that they can be collapsed axially.

In a further alternative arrangement (not shown) the sterile drape may be made from a relatively stiff material (for example paper), which may be originally folded and collapsed, then is expanded into the bore scanner for deployment. When expanded, the drape will take on a cylindrical shape matching that of the bore of the magnet.

In another embodiment for imaging systems with bores that do not have an inner surface which is entirely cylindrical, all the above mentioned techniques may be applied to drape a fraction of the bore. In particular, sections of the bore that acts as support surfaces for rolling patient support beds may need to remain clear and would not need to be sterile. In these configurations the bore drape may be applied only to a limited surface of the bore that needs to be maintained sterile, particularly the surface above and to the side of the patient, excluding the surfaces below the patient.

Some key features of the drape are as follows:

It extends all the way along the bore of the device.

It is cylindrical or partially cylindrical so that it follows the portion of the bore that is intended to be draped rather than the patient.

It is attached to the bore or the magnet rather than to the patient so that the patient can move independently of the drape or the magnet can move independently of the patient.

Since various modifications can be made in my invention as herein above described, and many apparently widely different embodiments of same made within the spirit and scope of the claims without department from such spirit and scope, it is intended that all matter contained in the accompanying specification shall be interpreted as illustrative only and not in a limiting sense.

1. A sterile drape assembly for use with a medical device having a cylindrical bore, the drape comprising:
a sheet of a surgical drape material;
a mounting arrangement for supporting the sheet in a generally cylindrical shape, or a part of a cylindrical shape, as a sleeve within the cylindrical bore of the device
the surgical drape material having the characteristics of being sterile and compatible with the medical device.

2. The sterile drape assembly according to claim 1 wherein the sleeve is shaped and arranged such that it is collapsed axially into an annular structure which can be deployed by expanding the structure axially along the bore.

3. The sterile drape assembly according to claim 2 wherein the annular structure is supplied in an annular container.

4. The sterile drape assembly according to claim 1 wherein the sleeve includes pull strings such that the sleeve is deployed by the pull strings extending along the bore.

5. The sterile drape assembly according to claim 2 wherein the sleeve includes a first end for engaging an end of the bore of the device and a second end for engaging a diagnostic table such that relative movement between the device and the diagnostic table acts to deploy the sleeve.

6. The sterile drape assembly according to claim 1 wherein there is provided a wire support which can be deployed axially and which provides a support for the sleeve.

7. The sterile drape assembly according to claim 1 wherein there is provided an axially extending arm such that the sleeve is deployed by axial extension of the arm.

8. The sterile drape assembly according to claim 1 wherein the sleeve includes adhesive strips along an outside surface of the sleeve for bonding to the bore, which strips are covered by cover strips and the cover strips are arranged such that longitudinal pulling of the cover strips acts to deploy the sleeve.

9. The sterile drape assembly according to claim 1 wherein the annular structure is supplied in an annular container and wherein the container forms part of an end collision detection system on an end of the device.

10. The sterile drape assembly according to claim 1 wherein the annular structure is supplied in an annular container and wherein the container is carried on a support not attached to the magnet but is available to be moved into place at the end of the device.

11. The sterile drape assembly according to claim 1 wherein the sleeve is shaped and arranged such that it is rolled longitudinally into a cylindrical structure with an axis which is arranged to extend along the bore and which can be deployed by expanding the structure outwardly into a cylinder shape.

12. The sterile drape assembly according to claim 11 wherein the sleeve is longitudinally rolled about a rod from which it can be then unrolled and pressed against the bore.

13. The sterile drape assembly according to claim 11 wherein the sheet is formed from a resilient sheet material which has sufficient resilience to expand from the initial rolled position to an expanded cylindrical condition.

14. The sterile drape assembly according to claim 1 wherein the sleeve is formed from a soft, pliable material that includes at least one chamber in the sleeve, which may then be inflated to produce a semi-rigid cylinder.

15. The sterile drape assembly according to claim 1 wherein the sleeve is formed from a soft, pliable material that includes at least one chamber in the sleeve; with the chambers being filled with objects arranged such that, when a vacuum is applied, the sleeve produces a semi-rigid structure.

16. The sterile drape assembly according to claim 1 wherein there is provided a cylindrical support member arranged to be mounted within the bore for supporting the sleeve.

17. The sterile drape assembly according to claim 16 wherein the cylindrical support member is hollow and includes a vacuum line which is connected to a hollow interior of the support member for drawing the sleeve against a perforated inner surface of the support member.

18. The sterile drape assembly according to claim 16 wherein the support member is arranged to engage the sleeve by an elastic nature of the sheet which is wrapped around the support member.

19. The sterile drape assembly according to claim 16 wherein the cylindrical support member is defined by a series of spaced members.

20. The sterile drape assembly according to claim 16 wherein the cylindrical support member includes a plurality of sections at axially spaced locations which can expand axially by relative movement of the sections.

21. The sterile drape assembly according to claim 16 wherein the cylindrical support member includes two support components each associated with a respective end of the bore for insertion into the respective end.

22. The sterile drape according to claim 1 wherein the elastic sheet is attached directly to the front and back faces of the device, through adhesives or clips.

23. The sterile drape assembly according to claim 1 wherein the sleeve is formed from a stiff material which is shaped and arranged such that it is supplied in an originally folded and collapsed condition from which it can be expanded to form the cylindrical shape by unfolding.

24. The sterile drape assembly according to claim 1 wherein the sleeve includes a plurality of overlying layers that may be peeled away one after the other to reveal the next underlying layer after a first is used.

25. The sterile drape assembly according to claim 1 wherein the sleeve is formed from a first material which is relatively stiff to conform to cylindrical portions of the bore and includes portions of a more flexible material which accommodate projections and/or recesses in the bore.

26. The sterile drape assembly according to claim 1 wherein the sleeve has circular portions defining sealed ends of the sleeve.

27. The sterile drape assembly according to claim 26 wherein the sealed ends are arranged to be removable prior to introduction of the patient into the bore.

28. The sterile drape assembly according to claim 26 wherein the sealed ends may be reapplied to the drape using adhesive, following patient removal from the bore to maintain sterility between imaging sessions of the same patient.

29. The sterile drape assembly according to claim 26 wherein the sealed ends are formed in separate pieces which can be folded back around the sleeve to engage and cover an end face of the device.
